(19) United States
(12) Plant Patent Application Publication
(10) Pub. No.: US 2013/0276182 P1
(43) Pub. Date: Oct. 17, 2013

(54) Strawberry Plant Named 'DrisStrawTwentyNine'

(50) ;

(71) Applicants: Carlos D. Fear, West Malling (GB); Matthias D. Vitten, Aptos, CA (US); Michael D. Ferguson, Moorpark, CA (US)

(72) Inventors: Carlos D. Fear, West Malling (GB); Matthias D. Vitten, Aptos, CA (US); Michael D. Ferguson, Moorpark, CA (US)

(73) Assignee: DRISCOLL STRAWBERRY ASSOCIATES, INC., WATSONVILLE, CA (US)

(21) Appl. No.: 13/506,409

(22) Filed: Apr. 17, 2012

(51) Int. Cl. A01H 5/00

(52) U.S. Cl. PLT/208

(57) ABSTRACT

A new and distinct variety of strawberry plant named ‘DrisStrawTwentyNine’ particularly characterized by having large fruit with a long shelf life, and high production of first class fruit, is disclosed.

### GENUS AND SPECIES

Fragaria×ananassa

### VARIETY DENOMINATION

‘DrisStrawTwentyNine’

### BACKGROUND OF THE NEW PLANT

The present invention relates to a new and distinct strawberry variety designated ‘DrisStrawTwentyNine’ and botanically known as Fragaria×ananassa. This new strawberry variety was discovered in Avitorejo, Spain in February 2007 and originated from a cross between the proprietary female parent ‘DrisStrawEight’ (U.S. Plant Pat. No. 20,735) and the proprietary male parent ‘274’ (unpatented). A single plant was selected for asexual propagation via tissue culture and vegetative cuttings in Palencia, Spain in 2007.

‘DrisStrawTwentyNine’ underwent further testing in Spain for five years (2007-2011). The present invention has been found to retain its distinctive characteristics through successive asexual propagations via stolons and tissue culture.

Plant Breeder's Rights for this variety have not been applied for. ‘DrisStrawTwentyNine’ has not been made publicly available or sold more than one year prior to the filing date of this application.

### SUMMARY OF THE INVENTION

The following are the most outstanding and distinguishing characteristics of this new cultivar when grown under normal horticultural practices in Avitorejo, Spain.

1. Long shelf life of fruit;

2. Large fruit; and

3. High production of first class fruit.

### DESCRIPTION OF THE PHOTOGRAPHS

The accompanying color photographs show typical specimens of the new variety at various stages of development. The colors shown are as true as can be reasonably obtained by conventional photographic procedures. The photographs were taken from six-month-old plants.

FIG. 1 shows overall plant habit including fruit at various stages of development.

FIG. 2 shows upper and lower surfaces of the leaves of the plant with three leaflets.

FIG. 3 shows both upper and lower surfaces of the flowers.

FIG. 4 shows the whole fruit.

FIG. 5 shows the fruit in longitudinal cross-section.

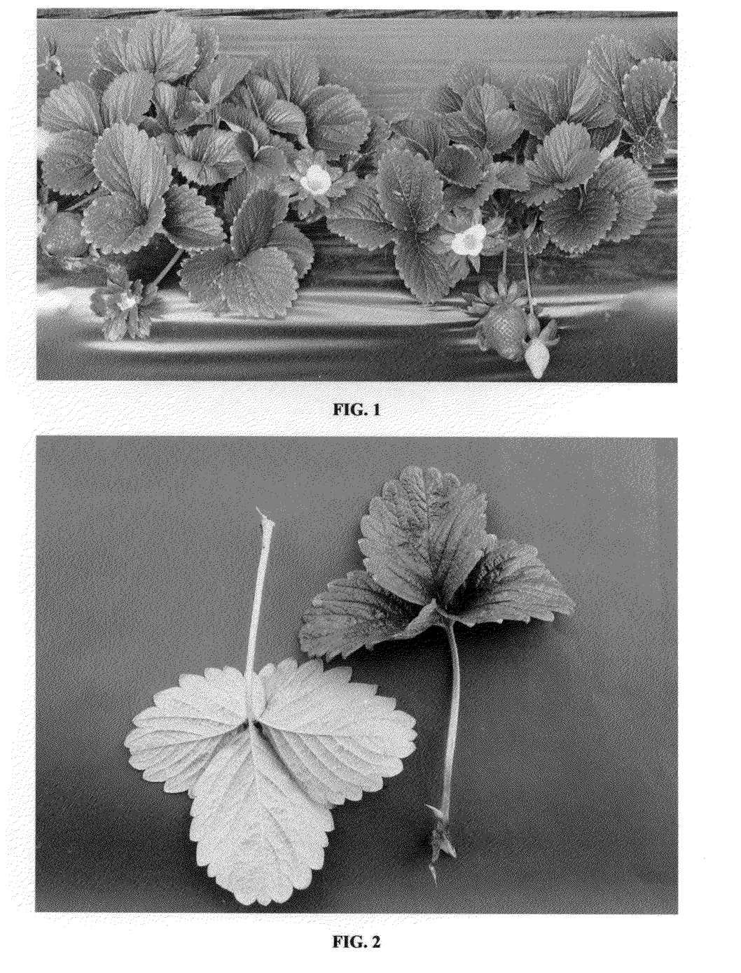

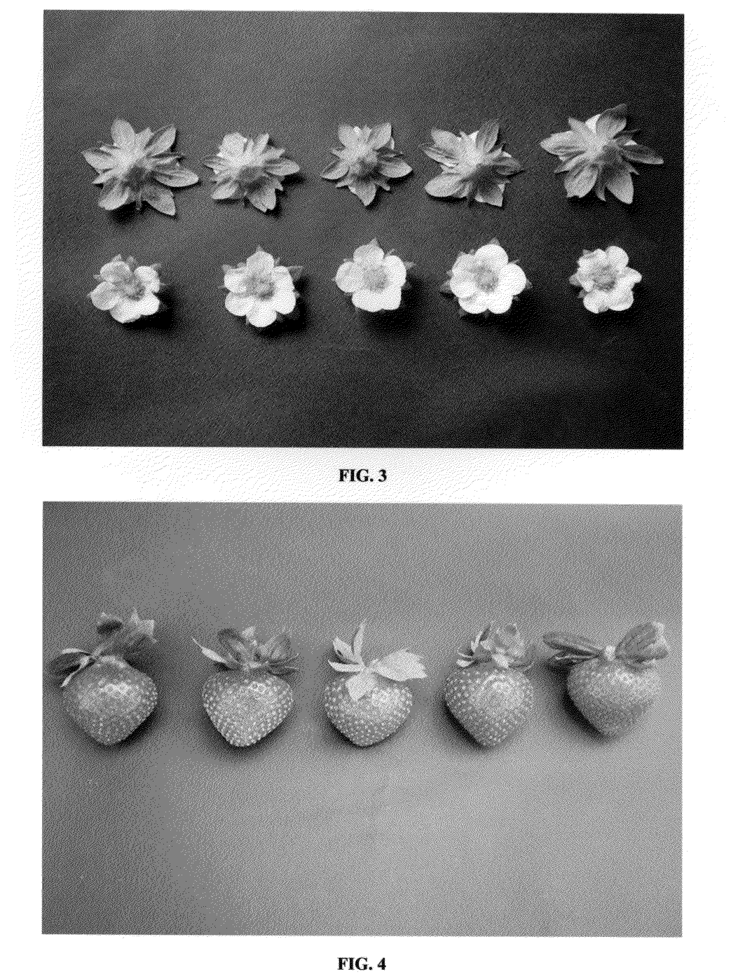

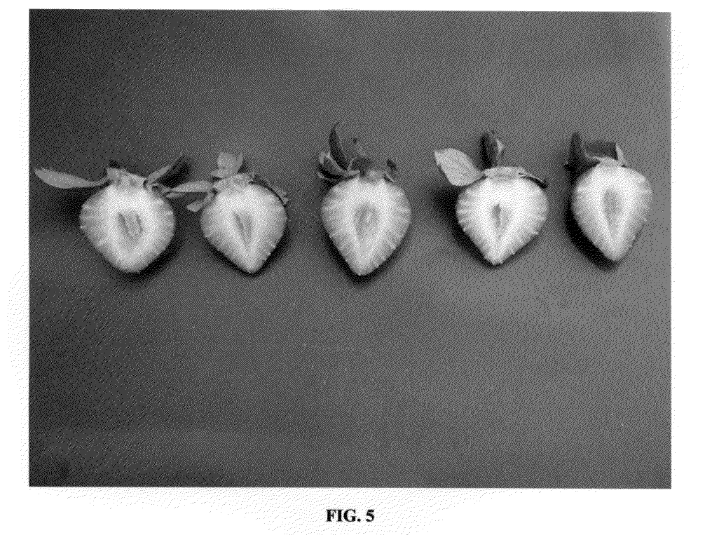

### DESCRIPTION OF THE NEW VARIETY

The following detailed descriptions set forth the distinctive characteristics of ‘DrisStrawTwentyNine’. The data which define these characteristics is based on observations taken in Avitorejo, Spain from 2010 to 2011. This description is in accordance with UPOV terminology. Color designations, color descriptions, and other phenotypical descriptions may deviate from the stated values and descriptions depending upon variation in environmental, seasonal, climatic, and cultural conditions. ‘DrisStrawTwentyNine’ has not been observed under all possible environmental conditions. The botanical description of ‘DrisStrawTwentyNine’ was taken from six-month-old plants. Color references are primarily to the R.H.S. Colour Chart of The Royal Horticultural Society of London (R.H.S.) (2007 edition). Descriptive terminology follows the Plant Identification Terminology, An Illustrated Glossary, 2^{nd} edition by James G. Harris and Melinda Woolf Harris, unless where otherwise defined.

### DETAILED BOTANICAL DESCRIPTION OF THE PLANT

Classification:
Species: Fragaria×ananassa
Common name: Strawberry
Denomination: ‘DrisStrawTwentyNine’
Parentage:
Female parent: The proprietary variety ‘DrisStrawEight’ (U.S. Plant Pat. No. 20,735)
Male parent: The proprietary variety ‘274’ (unpatented)
Plant:
Height: 39.3 cm
Diameter: 44.4 cm
Number of crowns/plant: 2
Habit: Flat globose
Density of individual plant: Open to sparse
Vigor (health and hardiness of plant): Strong
Terminal leaflets:
Size: Large
   Length.—10.75 cm.
   Width.—9.54 cm.
   Length/width ratio.—1.1 (Longer than broad).
Number of teeth/terminal leaflet: 18
Shape of teeth: Obtuse — serrate to crenate
Color:
   Upper surface.—RHS N137B (Dark green).
   Lower surface.—RHS 191A (Medium greyed-green).
Shape in cross section: Slightly concave to concave
Blistering: Medium
Glossiness: Strong
Number of leaflets: Three only
Shape: Orbicular
Base shape: Acute
Apex descriptor: Rounded
Variegation: Absent
Margin: Serrate
Margin profile: Flat (Level with the leaflet blade)
Petiole:
Length: 28.8 cm
Diameter: 4.37 mm
Pubescence: Sparse
Pose of hairs: Upwards
Color: RHS N144D (Light yellow-green)
Petiolule:
Length: 16.45 mm
Diameter: 2.87 mm
Bract frequency: 0
Color: RHS 144D (Light yellow-green)
Stipule:
Length: 4.2 cm
Width: 8.26 mm
Pubescence: Sparse
Stipule anthocyanin coloration: Absent or very weak; RHS 144B (Medium yellow-green)
Stolon anthocyanin coloration: Weak; RHS 144C (Light yellow-green)
Inflorescence:
Position relative to foliage: Above
Number of flowers: Few
Time of flowering (50% of plants at first flower): Early
Flower size: Large
Diameter: 26.92 mm
Petals:
   Shape.—Orbicular.
   Apex.—Rounded.
   Base.—Rounded.
   Margin.—Entire.
   Spacing.—Overlapping.
   Length.—13.76 mm.
   Width.—13.49 mm.
   Length/width ratio.—1.0 (As long as broad).
   Typical and observed petal number per flower.—6.
   Color (both upper and lower surfaces).—RHS 155C (White).
Calyx:
   Diameter.—43.43 mm.
   Diameter relative to corolla.—Much larger.
   Inner calyx diameter relative to outer.—Smaller.
   Insertion of calyx.—Level.
   Pose of calyx segments.—Spreading — outwards.
   Size of calyx in relation to fruit.—Slightly larger.
   Adherence of calyx.—Strong.
Sepal:
   Shape.—Oval to elliptical.
   Apex.—Truncate.
   Margin.—Entire.
   Length.—19.04 mm.
   Width.—10.07 mm.
   Typical and observed sepal number per flower.—6.
Receptacle color: RHS 151D (Light yellow-green)
Stamen: Present
   Anther color.—RHS 153C (Medium yellow-green).
Pedicel:
   Attitude of hairs.—Upwards.
Fruiting truss:
Length: Long; 38.9 cm
Diameter at base of truss: 5.21 mm
Number of berries per fruiting truss: 4
Attitude at first picking: Erect
Color at base of truss: RHS 144B (Medium yellow-green)
Fruit:
Relative fruit size: Large
Length: 43.12 mm
Width: 40.80 mm
Length/width ratio: 1.1 (As long as broad)
Fruit hollow length: 33.73 mm
Fruit hollow width: 18.40 mm
Fruit hollow length/width ratio: 1.8 (Longer than broad)
Fruit hollow center (cavity): Large
Weight (per individual berry): 30.2 g
Predominant fruit shape: Conical
Difference in shape between primary and secondary fruits: None or very slight (mid-season)
Evenness of fruit surface: Even or very slightly uneven
Fruit skin color: RHS 46B (Dark red)
Evenness of fruit color: Even or very slightly uneven
Fruit glossiness: Strong
Achenes:
   Insertion of achenes.—Level with surface.
   Coloration (sunward side of berry).—RHS 176A (Dark greyed-orange).
   Coloration (shaded side of berry).—RHS 172B (Medium greyed-orange).
   Number per berry.—344.2.
   Weight (weight of achenes divided by total # seed).—0.00067 g.
   Width of band without achenes.—Absent or very narrow.
Firmness of flesh (when fully ripe): Soft
Color of flesh (excluding core): RHS 44A (Medium red)
Color of core: RHS 33D (Light orange-red)
Evenness of flesh color: Even
Distribution of flesh color: Marginal and central
Sweetness: Medium
Acidity: Weak
Texture when tasted: Fine
Type of bearing: Not everbearing — not remontant
Grams of fruit/plant: 750.0. g
Harvest interval: Late December — late May
Harvest maturity: Early
Disease, pest, and stress resistance:
Botrytis fruit rot: Moderately resistant
Powdery mildew: Moderately susceptible
Drought: Moderately susceptible
High temperatures: Moderately susceptible
Wind: Moderately susceptible

### Comparison with Parental and Commercial Varieties

When ‘DrisStrawTwentyNine’ is compared to the proprietary female parent ‘DrisStrawEight’ (U.S. Plant Pat. No. 20,735), ‘DrisStrawTwentyNine’ has a better shelf life, higher yields, and higher percent of first class fruit at harvest than ‘DrisStrawEight.’ Additionally, ‘DrisStrawTwentyNine’ has slightly lower flavor scores than ‘DrisStrawEight’.

When ‘DrisStrawTwentyNine’ is compared to the commercial variety ‘DrisStrawSixteen’ (U.S. Plant Pat. No. 22,247), ‘DrisStrawTwentyNine’ has fruit with dark red skin color, a flat terminal leaflet margin profile, and a slightly concave to concave leaf shape in cross section, whereas ‘DrisStrawSixteen’ has fruit with medium red skin color, a revolute terminal leaflet margin profile, and a convex leaf shape in cross section. Additionally, ‘DrisStrawTwentyNine’ has none to very slight difference between primary and secondary fruits, whereas ‘DrisStrawSixteen’ has marked difference between primary and secondary fruits.

We claim:

1. A new and distinct variety of strawberry plant named ‘DrisStrawTwentyNine’ as described and shown herein.
